(19) United States
(12) Patent Application Publication
(10) Pub. No.: US 2003/0010675 A1
(43) Pub. Date: Jan. 16, 2003

(54) LIGHT OIL FOR REDUCED PARTICULATE EMISSION

(76) Inventors: TADAO OGAWA, AICHI (JP); MINORU YAMAMOTO, AICHI (JP); SHI-AKI HYODO, AICHI (JP); MASAMI YAMAMOTO, AICHI (JP); MASANORI OKADA, TOYOTA-SHI (JP); JUN-ICHI MATSUDAIRA, TOYOTA-SHI (JP); YOSHIO FUJIMOTO, TOYOTA-SHI (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/186,898

(22) Filed: Nov. 6, 1998

(30) Foreign Application Priority Data
Nov. 7, 1997 (JP) 9-306130

(51) Int. Cl. C07C 2/00

(52) U.S. Cl. 208/27; 585/734

(57) ABSTRACT

Light oil for reduced particulate emission which minimizes both the SOF and ISF of particulate matter emitted from a diesel engine is provided. The light oil for reduced particulate, (especially reduced SOF) emission comprises hydrocarbons, with the content of the distillation residue at 320° C. being 3% by volume or less in a distillation test according to ASTMD86-90. The light oil for reduced particulate (especially reduced soot) emission comprises straight chain paraffin, wherein when hydrocarbons except straight chain paraffin are mainly composed of branched chain paraffin and/or naphthene, their contents are 2% by volume or less, and when hydrocarbons except straight chain paraffin in the fuel are mainly composed of aromatics, their contents are 1% by volume or less.

### BACKGROUND OF THE INVENTION

1. Field of the Invention

This invention relates to light oil for reduced or low particulate emission which can reduce emissions of particulates (particulate matter (PM)) from a diesel engine without increasing NOₓ emissions.

2. Description of the Related Art

Regulations have been enforced on emissions from diesel engines such as PM, unburned hydrocarbons, and smoke. PM comprises substances derived from fuel and substances derived from lubricant oil.

In 1970's, it was announced that PM emissions from diesel engines and so on include carcinogens. Since then, a lot of studies have been published on the relationship between PM emissions and light oil characteristics. The light oil characteristics which have been examined for the past twenty years are of about ten kinds, including density, viscosity, 90% distillation temperature, aromatic contents and the cetane number.

Among these characteristics, it has been admitted that PM emissions are correlated, to some extent, with 90% distillation temperature (K. Tsurutani, Y. Takei, Y. Fujimoto, J. Matsudaira and M. Kumamoto, SAE952349), or density (S. A. Floysand, F. Kvinge and W. E. Betts, SAE932683). In addition, it has been reported that there is some correlation between PM emissions and polycyclic aromatic contents (C. Betroli, N. Del Giacomo, B. Iorio and M. V. Prati, SAE932733).

However, with regards to light oil characteristics, no critical parameter predicting PM emissions has been reported. Accordingly, no specification of light oil for reduced particulate emission has been reported.

### SUMMARY OF THE INVENTION

One of the reasons why no parameter effective in predicting PM emissions has been found is that sufficient consideration has not been given to the meaning and effect of the researched light oil characteristics have in PM generation processes.

Another reason is that extremely few studies have been made on the processes in which PM is generated from light oil in view of light oil composition.

PM derived from light oil comprises substances which are light oil emitted unreacted, substances emitted in the middle of reaction processes, and substances emitted after reactions are completed. The substances constituting PM can be divided into a soluble organic fraction (hereinafter referred to as SOF) and an insoluble organic fraction (hereinafter referred to as ISF) based on solubility in dichloromethane.

It is an object of the present invention to provide light oil for reduced particulate emission which minimizes both SOF and ISF emissions from diesel engines, by making a detailed study of generation processes of substances constituting PM.

As mentioned above, particulate matter (PM) comprises a fraction soluble in an organic solvent (SOF) and a fraction insoluble in an organic solvent (ISF). Studies of the present inventors have shown that generation processes of these fractions greatly differ from each other. On the base of this, light oil for reduced particulate emission of the present invention have the following two aspects.

The first aspect of the present invention is to provide light oil for reduced particulate emission to limit high boiling components of light oil in order to reduce SOF emissions.

The light oil for reduced particulate emissions according to the first aspect comprises hydrocarbons, wherein the contents of undistilled components at 320° C. in a distillation test according to ASTMD86-90 are 3% by volume or less.

The second aspect of the present invention is to provide light oil for reduced particulate emission, which is mainly composed of straight chain paraffin in order to reduce soot emissions.

The light oil for reduced particulate emissions according to the second aspect comprises hydrocarbons mainly composed straight chain paraffin, wherein the hydrocarbons except the straight chain paraffin comprise 2% by volume or less of branched chain paraffin and/or naphthene, or the hydrocarbons except the straight chain paraffin comprise 1% by volume or less of aromatic hydrocarbons.

Light oil which fulfills both the first and second aspects can reduce both a soluble organic faction and soot in particulates at the same time.

That is a light oil for reduced particulate emissions according to claim 2, wherein the contents of undistilled components at 320° C. in a distillation test according to ASTMD86-90 are 3% by volume or less, thereby reducing both a soluble organic fraction and soot in particulates.

It is preferable that the aforementioned hydrocarbons are mainly composed of straight chain paraffin having 18 or less carbon atoms.

Preferably, the aforementioned straight chain paraffin has 18 or less carbon atoms, and more preferably, 8 to 18 carbon atoms.

In order that no components are collected by a PM filter, it is at least necessary that the aforementioned straight chain paraffin is to be distilled at distillation temperatures of 320° C. or less in a distillation test defined by ASTMD86-90. However, as straight chain paraffin has a greater chain length, the straight chain paraffin is more easily crystallized. Therefore, it is preferable to constitute light oil with the aforementioned straight chain paraffin having 18 or less carbon atoms, which is in a liquid state at 280° C.

The aforementioned straight chain paraffin may be a mixture of parts of straight chain paraffin having 8 to 18 carbon atoms (for example, pentadecane and dodecane) because the fluidity is increased by mixing.

Note that, with respect to the aforementioned light oil, sulfur derived mainly from crude oil is not taken into consideration.

Manufacturing the light oil low particulate emissions according to the present invention can be achieved by distillating a raw material composed mainly of straight chain paraffin, for example, a paraffin-rich raw material synthesized from natural gas by Fischer-Trosch process, in the distillation temperature range up to 320° C. It is necessary to apply pretreatment for removing high boiling components of the raw material or pretreatment for removing components except straight chain paraffin, if light oil obtained by that distillation has one of the following features: (1) The contents of undistilled components at 320° C. in a distillation test according to ASTMD86-90 are more than 3% by volume. (2) When hydrocarbons except straight chain paraffin are mainly composed of aromatic hydrocarbons, their contents are more than 1% by volume. (3) When hydrocarbons except straight chain paraffin are mainly composed of branched chain paraffin and/or naphthene, their contents are more than 2% by volume.

The contents of components except straight chain paraffin in light oil can be determined by the following processes: First, light oil is separated into aliphatic hydrocarbons and aromatic hydrocarbons by silica gel column chromatography. Second, the aliphatic hydrocarbon fraction is divided into straight chain paraffin and other aliphatic hydrocarbons by gas chromatography using a non-polar column. The contents of hydrocarbons except straight chain paraffin can be obtained from the sum of the contents of aliphatic hydrocarbons except straight chain paraffin determined by the gas chromatography, and the contents of aromatic hydrocarbons determined by the silica gel column chromatography.

### DETAILED DESCRIPTION OF THE INVENTION

First, the present inventors have carried out a detailed analysis on the composition of light oil. Based on its results, the present inventors have studied the meaning of the conventionally researched light oil characteristics in PM generation processes. Further, the present inventors have analyzed the composition of light oil, exhaust gases and PM, and clarified PM generation processes.

These studies have clarified the following:

As shown in a PM generation process chart of FIG. 1, particulate matter (PM) derived from light oil comprises unreacted substances which are light oil emitted without reaction, substances which are emitted in the middle of combustion processes (carbonized products and oxidation products), and substances which complete combustion reactions (soot, carbon dioxide gas, water, etc.).

Substances constituting PM can be divided into a soluble organic fraction (SOF) and an insoluble fraction (ISF) on the base of solubility in dichloromethane. Here, oxidation means oxidation reaction in the broad meaning and includes carbonization in which hydrocarbons release hydrogen to form polynuclear aromatics (hereinafter referred to as PNA) and soot. Of course, oxidation mentioned here includes oxidation reaction in the narrow meaning, in which hydrocarbons react with oxygen to form alcohol, aldehyde, organic acid, carbon dioxide gas, and/or water.

As shown in FIG. 1, SOF is high boiling components (substances collected by a filter at 51.7° C.) of unreacted hydrocarbons, partial oxides of hydrocarbons (e.g., alcohol, aldehyde, organic acid), and partial carbonized hydrocarbons (e.g., PNA).

ISF is PM excluding SOF. A main component of the ISF is soot which is generated by carbonization reaction in case of using low sulfur light oil. In addition, ISF includes PNA (highly-condensed dichloromethane-insoluble substances), which are carbonization intermediates, and sulfates formed by oxidation of sulfur compounds in light oil.

Considering the statements in [2] and [3], it is clear that generation processes of SOF and ISF are entirely different from each other.

FIG. 2 shows a graph in which the ordinate shows PM, SOF and ISF emissions and the abscissa shows the temperature of exhaust gases immediately after passing through an exhaust valve. This experiment was conducted by driving a test engine under 6 kinds of conditions of load and revolutional speed. (The load is a value when the maximum output at each revolutional speed is assumed as 100% and the revolutional speed is a value when the revolutional speed at the time when the test engine produces the maximum output is assumed as 100%). It is apparent from FIG. 2 that large amounts of SOF were generated under the condition of low load and low revolutional speed where the exhaust gas temperature was low, and that large amounts of ISF, as well as NOₓ were generated under the condition of high load and high revolutional speed where the exhaust gas temperature was high. This result is in agreement with the statement in [3].

As shown in a schematic diagram of FIG. 3, areas through which light oil injected into an engine cylinder passes on the way to be emitted from the cylinder can be roughly divided into six areas based on oxygen concentration and temperature. The area surrounded by the bold line in FIG. 3 shows the existence of fuel injected into the cylinder. FIG. 3 also shows the generation areas of SOF, ISF, and HC. Note that numbers in parentheses designate the amounts of emissions (g/kWh) when an engine is driven under the condition of 5% load and 60% revolutional speed, using light oil available on the market.

Area \mathring{1} is called “flame”. In this area, the temperature is high and oxidation is carried out. This area has a temperature around 2000K, and hydrocarbons entering this area are completely burned into carbon dioxide gas and water.

Area \mathring{2} exists inside the flame. In this area, the temperature is high owing to the heat of the flame, but oxygen is insufficient because oxygen has been consumed by the flame. This area has a high-temperature reduction atmosphere. Most hydrocarbons entering this area are smothered into soot (a main component of ISF).

Area \mathring{3} exists near the flame and is an area where oxygen are abundant. In this area, the temperature is not high enough to complete oxidation of hydrocarbons. Hydrocarbons in this area are changed into partial oxides such as alcohol, aldehyde, and organic acid.

Area \mathring{4} lies near Area \mathring{2} and is an area where oxygen is insufficient and the temperature is rather low. In this area, hydrocarbons are not completely carbonized because of a low speed of hydrocarbon carbonization. That is, polynuclear aromatics (PNA) are generated.

Area \mathring{5} exists near Area \mathring{3} and is an area where the temperature is lower than that of Area \mathring{3}. In this area, oxygen is abundant but oxidation reaction hardly proceeds because of a low temperature.

Area \mathring{6} lies near Area \mathring{4} and is an area where the temperature is lower than that of Area \mathring{4}. In this area, oxygen is insufficient but carbonization reaction hardly proceeds because of a low temperature.

Areas \mathring{5} and \mathring{6} are different from each other in oxygen concentration but hydrocarbons flowing through these areas are emitted unchanged due to low temperatures.

In view of the above, it is assumed that when fuel flows through Areas \mathring{3} and \mathring{4}, differences in stability and reactability of hydrocarbons remarkably appear. That is to say, it is considered that hydrocarbons which are not easily burned have a high probability of being emitted unburned and a high probability of being carbonized.

It is known that PM emissions are largely influenced by load on an engine and are inversely proportional to NO emissions. This invention aims to provide a light oil composition for reducing PM emissions without increasing NOₓ emissions.

The light oil for reduced particulate emissions according to the present invention has been attained based on the above findings. The present inventive light oil has two aspects:

The first aspect of the present invention is to provide light oil which contains no high boiling hydrocarbons, which are to be collected by a PM filter, even when emitted without reaction.

The present inventors have found through their experiments that hydrocarbons to be collected by a filter at 51.7° C. are components remaining in a distillation still at 320° C. in a distillation test according to ASTMD86-90. The content of this residue is hardly set without engine driving conditions or regulated PM emissions. In the present invention, the content of distillation residue has been set based on the following results.

The percentage of hydrocarbons emitted unreacted to fuel injected into an engine cylinder (fuel consumption) was about 2% under the condition where an engine was driven idly and about 0.2% under the condition where the engine was driven under 80% load.

On the other hand, the percentage of distillation residue of the tested light oils at 320° C. ranged from 3 to 26%. The comparison of these results indicates that not all high boiling components of light oil are emitted without reaction.

From the above results, in the present invention, the contents of high boiling point components in light oil, i.e., the content of distillation residue at a distillation temperature of 320° C. is set to 3% or less.

The second aspect of the present invention is to provide light oil constituted with hydrocarbons having the remotest relationship with soot generation where the ratio of hydrogen to carbon approximately equals 0, i.e., paraffin, which is saturated. Moreover, the second aspect is to provide light oil with more flammable straight chain paraffin than other paraffin.

More concretely, when hydrocarbons except straight chain paraffin are branched chain paraffin and/or naphthene, their contents are set to 2% by volume or less, and when hydrocarbons except straight chain paraffin include aromatic hydrocarbons, their contents are set to 1% by volume or less.

By the way, results of the conventional distillation tests defined by ASTMD86-90, JIS K2254, etc. have been classified in view of the relationship between distillate percentage and temperature. The results have been evaluated by the temperature at which a predetermined percentage of distillate is obtained, as typically shown by 90% distillation temperature (T90). For example, it has been regarded that a high T90 value means that there are large amounts of high boiling components among components to be distilled.

So, the present inventors have employed distillation residue percentage at a distillation temperature corresponding to T80 to T90 as a value indicating directly the contents of high boiling components.

To be straight chain paraffin which is distilled by 320° C. in the aforementioned distillation test satisfies the condition of hydrocarbons which easily pass through a PM filter and which are very flammable. When passing through Areas \mathring{3} and \mathring{4} of FIG. 3, such paraffin has a high probability of being emitted after completing combustion reaction and a low probability of being carbonized. Accordingly, SOF and ISF are suppressed from being generated. In other words, the amount of PM generated can be decreased.

Straight chain paraffin which is distilled by 320° C. in that distillation test is, for example, paraffinic hydrocarbons having 18 or less carbon atoms, in view of the boiling points of hydrocarbons shown in Table 1. Among them, mixtures of straight chain paraffin having 8 to 18 carbon atoms, such as octane, nonane, decane, undecane, dodecane, tridecane, tetradecane, pentadecane, hexadecane, heptadecane and octadecane are preferable in view of combustibility and a high engine output power. It is more preferable to use a mixture of pentadecane and decane.

TABLE 1

| | Carbon | Melting Point | Boiling Point |
|---|---|---|---|
| | Number | (° C.) | (° C.) |
| methane | 1 | −182.5 | −161.5 |
| ethane | 2 | −183.3 | −88.6 |
| propane | 3 | −187.7 | −42.1 |
| butane | 4 | −138.4 | −0.50 |
| pentane | 5 | −129.8 | 36.1 |
| hexane | 6 | −95.3 | 68.7 |
| heptane | 7 | −90.6 | 98.4 |
| octane | 8 | −56.8 | 125.7 |
| nonane | 9 | −53.5 | 150.8 |
| decane | 10 | −29.7 | 174.1 |
| undecane | 11 | −25.6 | 195.9 |
| dodecane | 12 | −9.7 | 216.2 |
| tridecane | 13 | −6 | 234 |
| tetradecane | 14 | 5.5 | 251 |
| pentadecane | 15 | 10 | 268 |
| hexadecane(cetane) | 16 | 18.2 | 287.1 |
| heptadecane | 17 | 22.0 | 303 |
| octadecane | 18 | 28.0 | 308 |
| nonadecane | 19 | 32 | 330 |
| eicodecane | 20 | 36.6 | 345.12 |
| heneicosane | 21 | 40.4 | 215 (l5 mmHg) |
| docosane | 22 | 44.4 | 224 (l5 mmHg) |
| tridocosane | 23 | 47.4 | 234 (15 mmHg) |
| tetracosane | 24 | 51.1 | 240 (l5 mmHg) |
| pentacosane | 25 | 53.3 | 259 (l5 mmHg) |

It is preferable that the entire volume of fuel to be tested is distilled at 320° C. in the aforementioned distillation test. However, considering distillation test reproductivity, dispersion, and the fact that not all distillation residue at 320° C. is emitted without reaction, it is necessary to set the amount of distillation residue at 320° C. to be 3% or less. More distillation residue than this is not preferable, because the aiming reduction of PM emissions, particulary, of SOF emissions cannot be achieved.

The content of hydrocarbons except straight chain paraffin contained in the residue distillated at 320° C. must be set to 2% by volume or less when the fuel to be distilled contains no aromatic hydrocarbons, or must be set to 1% by volume or less when the fuel to be distilled contains aromatic hydrocarbons.

The reason why the contents of hydrocarbons except straight chain paraffin are set in different ranges in accordance with the composition is that those materials have different combustibilities in accordance with different structures of branched chain paraffin, naphthene, and aromatic hydrocarbons. This will be described in detail in the preferred embodiments of the present invention.

The present invention has the following advantages.

According to the present invention, by setting a distillation end point at 320° C. or less, SOF emissions derived from unreacted light oil can be minimized, and by restricting the contents of hydrocarbons except straight chain paraffin, soot generated by incomplete combustion and carbonization can be minimized. By satisfying these two requirements, it is possible to minimize not only particulate emissions but also emissions of unreacted hydrocarbons, black smoke and NOₓ

### BRIEF DESCRIPTION OF THE DRAWINGS

The exact nature of this invention, as well as other objects and advantages thereof, will be readily apparent from consideration of the following specification relating to the annexed drawings, in which:

FIG. 1 is a chart for explaining PM generation processes;

FIG. 2 is a graph showing the relationship between exhaust gas temperature and SOF emissions or ISF emissions;

FIG. 3 is a schematic diagram for showing areas where PM, reacted hydrocarbons, carbon dioxide gas, and water are generated by the combustion of fuel;

FIG. 4(a) is a graph showing a comparison of carbon number distributions of straight chain paraffin which are components of fuel and SOF;

FIG. 4(b) is a graph showing carbon number distributions of fuel, and fuel distillation residues at 310° C. and 320° C.;

FIG. 5(a) is a graph showing the relationship between the content of high boiling components of light oil, and SOF emissions;

FIG. 5(b) is a graph showing the relationship between the hydrocarbon contents from exhaust gases multiplied by distillation residue ratios at 320° C. and SOF emissions.

FIG. 6 is a graph showing a light oil composition map and boiling points of straight chain paraffin; and

FIG. 7 is a gas chromatogram of light oil available on the market.

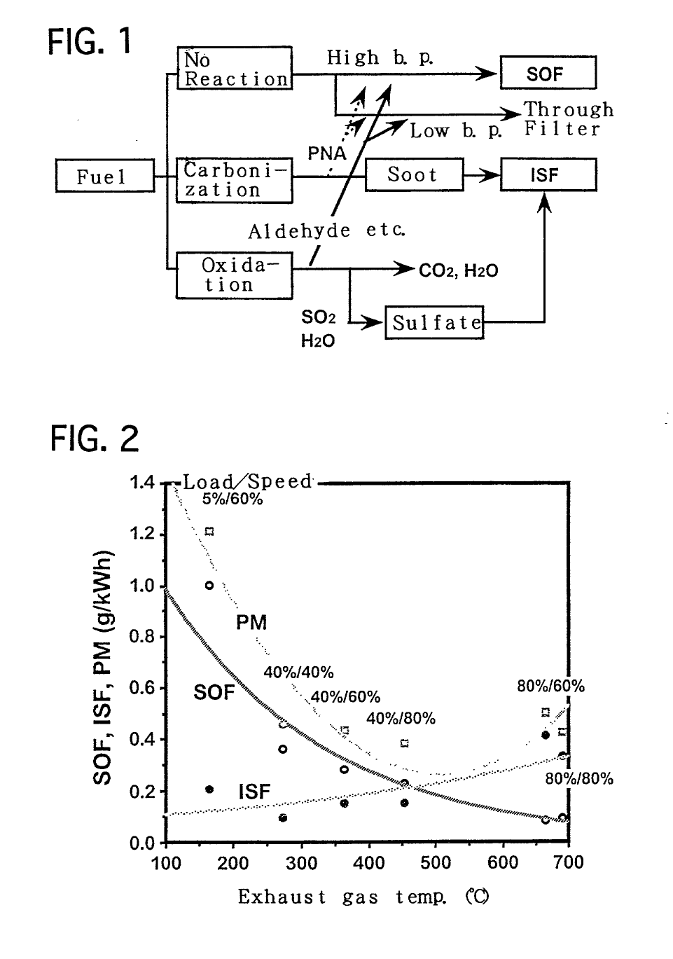

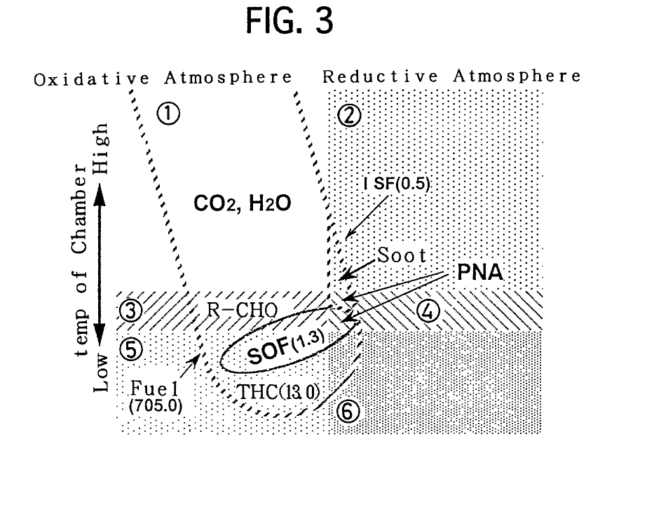

### DETAILED DESCRIPTION OF THE PREFERRED EMBODIMENTS

The present invention will be concretely described hereinafter.

As mentioned before, substances constituting PM are divided into SOF and ISF depending on the solubility in dichloromethane. SOF is dichloromethane extracts existing in exhaust gases and collected by a PM filter at 51.70° C., i.e., constituting PM.

Concretely, SOF comprises unreacted light oil, partially oxidized light oil and partially carbonized light oil material (lowly-condensed aromatic hydrocarbons). Of all these substances, only high boiling components are trapped by the above PM filter. Therefore, as a method for reducing (decreasing) SOF, two methods are conceivable: one is a method for reducing (decreasing) high boiling components of light oil and the other is a method for reducing (decreasing) hydrocarbons which are emitted unreacted, partially oxidated or partially carbonized

The first method is a method for minimizing SOF derived from unreacted light oil, that is to say, a method for removing completely, from light oil, high boiling light oil components to be collected by a PM filter at 51.7° C.

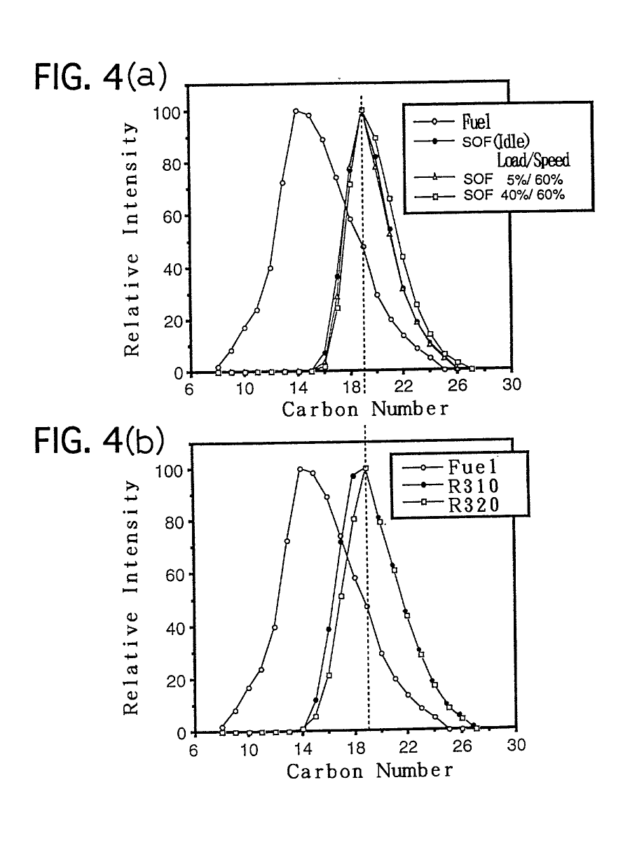

SOF emitted from a diesel engine was analyzed by gas chromatography. The determined peak intensity of straight chain paraffin relative to most intense peak is plotted against its carbon number in FIG. 4(a). FIG. 4(a) shows carbon number distributions of fuel and SOF emitted under the conditions in which an engine is driven idly, under a low load, or under a high load. It is apparent from FIG. 4(a) that the SOF contains components having 16 or more carbon atoms.

The same light oil as supplied to the engine in FIG. 4(a) was distilled by a temperature of 310° C. or 320° C. The distillation residue remaining in a distillation flask was collected and their carbon numbers were determined in the same way as in the experiment shown in FIG. 4(a). FIG. 4(b) shows distributions of the determined carbon numbers.

FIG. 4(b) demonstrates that the residue at a distillation temperature of 310° C. contains larger amounts of components having small carbon numbers than the residue at a distillation temperature of 320° C. Hydrocarbons having small carbon numbers shown by the distillation residue at 310° C. were not seen in the SOF shown in FIG. 4(a).

On the other hand, the carbon number distribution of the distillation residue at 320° C. was the same as those of the SOF shown in FIG. 4(a). This fact shows that when hydrocarbons which are distilled at 320° C. or more are emitted unreacted, they will be collected by a PM filter.

Six kinds of light oil having different distillation characteristics were supplied to a direct injection diesel engine and experiments were conducted on exhaust gases. Among exhaust gas experimental data, SOF emissions and unreacted hydrocarbon emissions were evaluated under the condition in which the engine was driven at a revolutional speed of 60% (the revolutional speed of 60% is 60% of the value when the revolutional speed at the time when the test engine produces the maximum output is assumed as 100%) under a load of 40% (the load of 40% is 40% of the value when the maximum output at each revolutional speed is assumed as 100%).

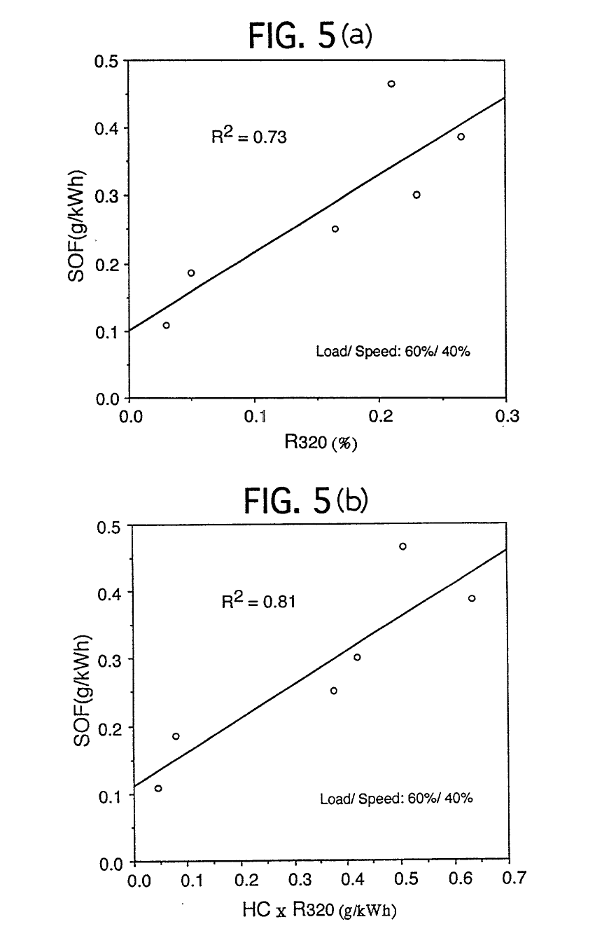

The relationship between the distillation residue ratios at 320° C. and SOF emissions on the six kinds of light oil were studied. It was found that as shown in the FIG. 5(a) the amount of SOF emissions is highly influenced by the contents of high boiling components in light oil.

The SOF, here, is a high boiling components in the unburned component (mainly unburned light oil) in the exhaust gas. Therefore, the relationship between SOF emissions and hydrocarbon contents in exhaust gas, which is measured with flame ioning action detector (FID) and so on, multiplied by distillation residue ratios at 320° C. (HCxR 320) must be examined. FIG. 5(b) shows the result that SOF emissions are highly correlated with the contents of high boiling hydrocarbons in the exhaust gas.

The hydrocarbon content in the exhaust gas is different depending on the composition of the various light oils even under the same engine condition. This is because the structure of the hydrocarbon has influence on HC emission in addition to the boiling point of the hydrocarbon in the exhaust gas. However, the SOF emissions become 0, if the hydrocarbon in the exhaust gas does not contain the high boiling components which are collected by a PM filter controlled at 51.7° C.

Thus, it has became apparent that one method of reducing SOF is to remove, from light oil, hydrocarbons which are distilled above 320° C.

In summary, the first method is to constitute hydrocarbons of light oil with a distillate at a temperature of 320° C. or less. Thereby, SOF emissions can be minimized. In consideration of dispersion in a distillation test, the content of distillation residue at 320° C. in hydrocarbons used in the distillation test is set to be 3% by volume or less.

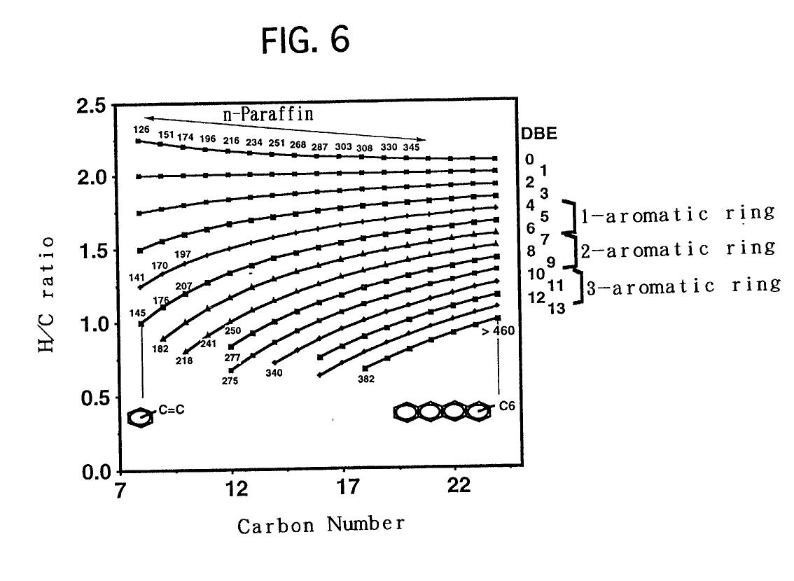

The second method is to prepare light oil having the minimum contents of hydrocarbons which are emitted without reaction or in the middle of combustion processes. In other words, this is to constitute light oil only with easily flammable hydrocarbons. A composition map of hydrocarbons constituting light oil is shown in FIG. 6.

In the map of FIG. 6, the ordinate shows the carbon number of hydrocarbons constituting light oil, while the abscissa shows the ratio of hydrogen atoms to carbon atoms of each hydrocarbon molecule. Thus, the composition of hydrocarbons contained in light oil can be shown.

As shown in FIG. 6, light oil contains hydrocarbons having the double bond equivalence value (referred to as DBE and shown by the right ordinate) of 0 (saturated hydrocarbons) to about 13.

The first step of combustion reaction of a hydrocarbon is a reaction of releasing hydrogen from the hydrocarbon and forming a hydrocarbon radical. Formability of this hydrocarbon radical is greatly influenced by stability of the hydrocarbon molecule.

The increase of DBE by one means elimination of one hydrogen molecule. This hydrogen elimination stabilizes the hydrocarbon. This is because an unsaturated bond (a double bond or a triple bond) or a ring structure is formed with the release of hydrogen. Conjugated olefin and aromatic rings, in which unsaturated bonds are conjugated, are especially stable because of a resonance structure given by a π electron. (Junichi Aihara “Why are Aromatic Compounds Stable?”, Science, June 1988)

This is supported by relative sensitivity of hydrocarbon molecular ions in electron ionization mass spectrometry. As shown in Table 1, it is known that molecular ion sensitivity is higher in the order of aromatic ring>saturated ring>conjugated olefin>alkane, and that as hydrocarbons have higher DBE, the hydrocarbons are stabler. (F. E. McLafferty, F. Turecek “Interpretation of Mass Spectra” 4th ed. (55D Gate Five Road Sausalito, Calif. 94965, USA: University Science Books, 1993))

| | Intensity of M⁺ peak relative to most intense peak (%) | | | | | |
|---|---|---|---|---|---|---|
| Compound type | M. W. ˜75 | % | M. W. ˜130 | % | M. W. ˜185 | % |
| Aromatic | 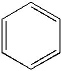 | 100 | 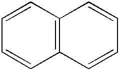 | 100 | 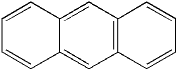 | 100 |
| Cycloalkane | 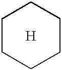 | 70 | 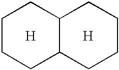 | 90 | 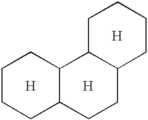 | 90 |
| Conjugated olef- | Hexatriene | 55 | all-Ocimene | 40 | | |
| in | | | | | | |
| Olefin | C₂ C═CC₂ | 35 | C₃ C═CC₄ | 20 | C₁₁C═C | 3 |
| | | | C₆ C═CC | 7 | | |
| Alkane | C₅ | 9 | C₉ | 6 | C₁₃ | 5 |
| Branched alkane | 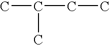 | 6 | (C₂)₂ CC₄ | 1 | (C₄)₃CH | 1 |
| | 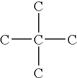 | 0.01 | 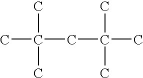 | 0.05 | 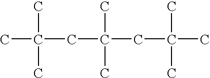 | 0.03 |

When straight chain alkane (i.e., straight chain paraffin) and branched chain alkane (i.e., branched chain paraffin) both having DBE of 0 are compared with each other, branched chain alkane is lower in molecular ion sensitivity than straight chain alkane. For example, when comparing hydrocarbons having 5 carbon atoms as shown in Table 2, molecular ion intensity of straight chain alkane is 9, but that of branched chain alkane having a secondary carbon is as small as 6 and that of branched chain alkane having a tertiary carbon is as small as 0.01. That is to say, it is shown that molecular ions of branched chain alkane are unstable. This is because a bond between a branched carbon atom in branched chain alkane and a carbon atom adjoining to that branched carbon atom is easily cut of f.

Table 3 shows frontier electron density as a parameter indicating reactivity of radicals obtained from straight chain alkane and branched chain alkane respectively. (Teijiro Kizu, Chikayoshi Nagata, Hiroshi Kato, Sen Imamura, Keiji Morokuma “Ryoshi Kagaku Nyumon (Quantum Chemistry for Beginners) 3rd Edition Vol.I” (Japan: Kagaku Dojin 1983) p. 245) It is apparent from Table 3 that a straight chain alkyl radical has the highest frontier electron density and the highest reactivity. It is also clear that hydrocarbons with more branches have lower frontier electron density and lower reactivity.

TABLE 3

| Chemical Compound | Electron Density | Orbital Energy |
|---|---|---|
| R— | (C_{Nr})² | λ_{N} |
| CH₃— | 0.8037 | −0.04150 |
| CH₃CH₂— | 0.7725 | −0.03173 |
| CH₃CH₂CH₂— | 0.7690 | −0.03084 |
| CH₃CH₂CH₂CH₂— | 0.7683 | −0.03076 |
| (CH₃)₂CH— | 0.7410 | −0.02193 |
| (CH₃CH₂)₂CH— | 0.7340 | −0.02021 |
| (CH₃)₃C— | 0.7094 | −0.01288 |

The above tendency is in agreement with a tendency of fuel evaluation using an engine for fuel evaluation, i.e., tendencies of hydrocarbons in the octane number and cetane number.

The octane number is used as an index for anti-knock quality of gasoline and a larger octane number indicates lower self ignitability and lower flammability.

In general, octane numbers of hydrocarbons have the following order:

aromatic hydrocarbons>olefin, naphthene≧brached chain paraffin>straight chain paraffin

In addition, the following facts are known from the octane number of paraffin and the octane number of aromatic hydrocarbons (See Takeshi Saito, ed. “Jidosha Kougaku Zensho (Automotive Engineering Encyclopedia), vol.7: Fuels and Lubricants for Automobiles” (Japan: Sankaido Co.) pp.69-70):

1. As the number of branches is larger, the octane number is larger (the hydrocarbon is less flammable).

2. As the length of carbon chain is greater, the octane number is smaller (the hydrocarbon is more flammable).

3. Hydrocarbons having a benzene ring have a high octane number (less flammable).

On the other hand, the cetane number of light oil indicates ignitability and flammability of hydrocarbons. Therefore, the cetane number and the octane number are exactly the opposite of each other with regard to combustibility of hydrocarbons. It is known that cetane numbers of hydrocarbons have the following order:

straight chain paraffin>branched chain paraffin>naphthene>olefin >aromatic hydrocarbons

It is known from the above tendency that the most flammable hydrocarbons among hydrocarbons contained in light oil is straight chain paraffin, which has a great chain length.

From the first and second methods, it is known that light oil constituted by straight chain paraffin which is distilled at a temperature of 320° C. or less in a distillation test can reduce the amount of PM generated.

[Reduction of Soot as a Component of ISF, and PNA]

Hydrocarbons constituting light oil comprise, as shown in FIG. 6, hydrocarbons having 8 to 24 carbon atoms and 0 to 13 DBE. These hydrocarbons make a dehydrogenation reaction under a reduction atmosphere at elevated temperatures. At the same time, the hydrocarbons make such a reaction as decomposition, cyclization, condensation, and aggregation. As a result, the hydrocarbons form PNA and are further carbonized to yield soot.

As apparent from FIG. 6, for example, in order that paraffin (DBE=0) becomes tetracyclic aromatics (DBE=13), a hydrogen molecule must be eliminated thirteen times.

From the above facts, it is clear that hydrocarbons having the remotest relationship with soot generation with regard to carbonization reaction (dehydrogenation), i.e., hydrocarbons which are most difficult to generate soot is paraffin, which has 0 DBE.

Between two kinds of paraffin (straight chain paraffin and branched chain paraffin), paraffin having less flammability and accordingly having a higher probability of passing through a reduction atmosphere at elevated temperatures is branched chain paraffin. Therefore, it is preferable to remove branched chain paraffin from light oil components.

Here, straight chain paraffin contained in light oil components has 8 to 24 carbon atoms. It is known that in general, not only to mention paraffin, as the alkyl carbon number in one molecule is greater, the cetane number is also greater.

Among hydrocarbons with the same DBE including paraffin which has 0 DBE, as the carbon number is larger, i.e., as hydrocarbons have alkyl groups at a higher ratio, the cetane number is greater.

On the other hand, with the increase of carbon number, straight chain paraffin has a higher boiling point and is more hardly vaporized. Accordingly, the probability of bonding with oxygen on a molecular level becomes lower. As a result, the straight chain paraffin is emitted unreacted, and unreacted hydrocarbon emissions and SOF emissions are increased.

It is concluded from the above that a light oil component which has the minimum soot emissions is straight chain paraffin having a greater chain length as far as it can be vaporized in an engine cylinder.

Therefore, it can be concluded that light oil with small SOF emissions is a mixture of hydrocarbons which are distilled at a temperature of 320° C. or less in a distillation test and composed only of very flammable hydrocarbons (straight chain paraffin).

It is also concluded that light oil with small PM emissions is a hydrocarbon mixture composed only of straight chain paraffin having 8 to 18 carbon atoms.

By satisfying the above conditions, this light oil can minimize not only PM emissions but also black smoke soot and unreacted hydrocarbon emissions.

In the meanwhile, it is demanded that diesel fuel should have a high fluidity in a fuel tank and pipes even in a cold area and be vaporized immediately after injected into an engine cylinder.

When straight chain paraffin which meets these demands is selected from Table 1 (melting points and boiling points of straight chain paraffin having 1 to 25 carbon atoms are shown), straight chain paraffin having 8 to 18 carbon atoms meets the demands.

For example, in an area where the lowest temperature is 6° C., straight chain paraffin having 12 or 13 carbon atoms meets the demands, and in an area where the lowest temperature is 28° C., straight chain paraffin having 17 or 18 carbon atoms meets the demands.

The contents of hydrocarbons except straight chain paraffin in straight chain paraffin are determined by the following process:

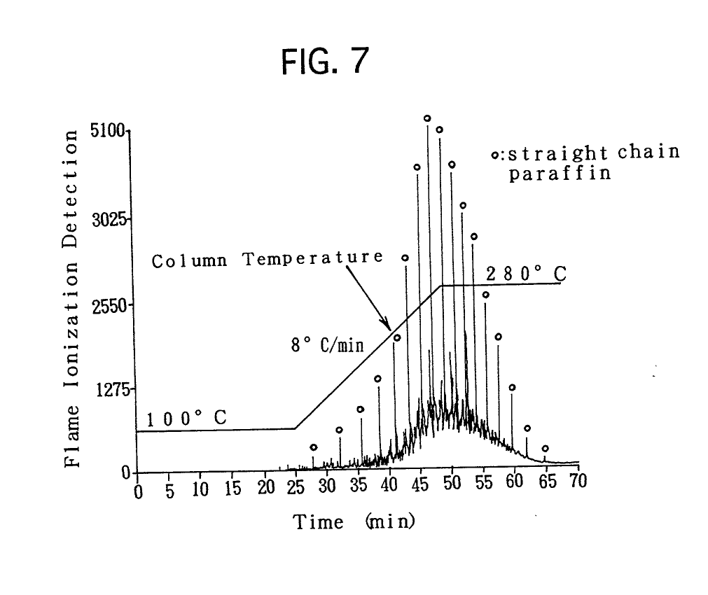

In general, diesel fuel is analyzed by liquid chromatography or gas chromatography. With regard to the present inventive fuel composed mainly of straight chain paraffin, gas chromatography using a nonpolar column is effective as a method of separating a fuel into straight chain paraffin and other hydrocarbons and determining each content. A gas chromatogram of the conventional light oil available on the market is shown in FIG. 7. In this figure, peaks with a circle are interpretable as peaks of straight chain paraffin. Peaks without circles in FIG. 7 are interpretable as those of aliphatic hydrocarbons except straight chain paraffin and aromatic hydrocarbons. There is a possibility that peaks with a circle are overlapped with peaks of aromatic hydrocarbons. Therefore, in order to determine strictly the contents of hydrocarbons except straight chain paraffin, it is necessary to separate a fuel by liquid chromatography beforehand into aliphatic hydrocarbons and aromatic hydrocarbons, and then conduct a gas chromatography of those aliphatic hydrocarbons, thereby determining the contents of hydrocarbons except the straight chain paraffin in the aliphatic fraction. When a gas chromatography is carried out on an aliphatic hydrocarbon fraction after separated by liquid chromatography, there will be an extremely low probability that some hydrocarbons except straight chain paraffin have the identical peaks with those of straight chain paraffin.

The overlapping of the peaks of straight chain paraffin and those of other aliphatic hydrocarbons can be confirmed by conducting mass spectrometry additionally. The content of straight chain paraffin can be strictly determined by being compared with a standard substance.

In the light oil according to the present invention, the contents of hydrocarbons other than straight chain paraffin determined by the aforementioned liquid chromatography and gas chromatography are set to be 2% by volume or less when containing no aromatic hydrocarbons and 1% by volume or less when containing aromatic hydrocarbons.

What is claimed is:

1. Light oil for reduced particulate emissions comprising hydrocarbons, wherein the contents of undistilled components at 320° C in a distillation test according to ASTMD86-90 are 3% by volume or less.

2. Light oil for reduced particulate emissions comprising: hydrocarbons mainly composed of straight chain paraffin, wherein
said hydrocarbons except said straight chain paraffin comprise 2% by volume or less of branched chain paraffin and/or naphthene, or
said hydrocarbons except said straight chain paraffin comprise 1% by volume or less of aromatic hydrocarbons.

3. Light oil for reduced particulate emissions according to claim 2, wherein
the contents of undistilled components at 320° C. in a distillation test according to ASTMD86-90 are 3% by volume or less,
thereby reducing both a soluble organic fraction and soot in particulates.

4. Light oil for reduced particulate emissions according to claim 1, wherein said hydrocarbons are mainly composed of straight chain paraffin having 18 or less carbon atoms.

5. Light oil for reduced particulate emissions according to claim 2, wherein said straight chain paraffin has 18 or less carbon atoms.

6. Light oil for reduced particulate emissions according to claim 2, wherein said straight chain paraffin has 8 to 18 carbon atoms.
